(19) United States
(12) Patent Application Publication
(10) Pub. No.: US 2018/0353524 A1
(43) Pub. Date: Dec. 13, 2018

(54) CANCER TREATMENT USING 2-DEOXY-2-FLUORO-L-FUCOSE IN COMBINATION WITH A CHECKPOINT INHIBITOR

(71) Applicant: SEATTLE GENETICS, INC., Bothell, WA (US)

(72) Inventors: Shyra Gardai, Bothell, WA (US); Che-Leung Law, Shoreline, WA (US); Peter Senter, Seattle, WA (US); Nicole Okeley, Redmond, WA (US); Jessica Field, San Francisco, CA (US)

(73) Assignee: SEATTLE GENETICS, INC., Bothell, WA (US)

(21) Appl. No.: 15/781,256

(22) PCT Filed: Dec. 2, 2016
(86) PCT No.: PCT/US16/64783

§ 371 Date: Jun. 4, 2018

Related U.S. Application Data
(60) Provisional application No. 62/263,228, filed on Dec. 4, 2015.
(60) Provisional application No. 62/308,583, filed on Mar. 15, 2016.
(60) Provisional application No. 62/321,857, filed on Apr. 13, 2016.

(51) Int. Cl. A61K 31/70; A61K 39/395; A61P 35/00

(52) U.S. Cl. CPC A61K 31/70; A61P 35/00; A61K 39/39558

(57) ABSTRACT

The present invention is directed to methods for treating cancer comprising administering to a subject in need thereof an effective amount of 2-deoxy-2-fluoro-L-fucose or a prodrug thereof, or a pharmaceutically acceptable salt thereof, in combination with a checkpoint inhibitor.

### CROSS-REFERENCES TO RELATED APPLICATIONS

This application claims the benefit of U.S. Provisional Patent Application Nos. 62/263,228, filed Dec. 4, 2015; 62/308,583, filed. Mar. 15, 2016; and 62/321,857, filed Apr. 13, 2016, each of which is incorporated herein in its entirety and for all purposes.

### BACKGROUND OF THE INVENTION

L-fucose, also referred to as 6-deoxy-L-galactose, is a monosaccharide that is a component of some N- and O-linked glycans and glycolipids in animals. (See Becker and Lowe, Glycobiology 13:41R-51R (2003).) Fucose is typically added as a terminal modification to glycans, including glycans attached to blood group antigens, selectins and antibodies. Fucose can be attached to glycans via α(1,2)-, α(1,3)-, α(1,4)- and α(1,6)-linkages by specific fucosyltransferases. α(1,2)-fucose linkages are typically associated with the H-blood group antigens. α(1,3)- and α(1,4)-fucose linkages are associated with modification of Lewis^{X} antigens. α(1,6)-fucose linkages are associated with N-linked GlcNAc molecules, such as those on antibodies.

Fucosylation of proteins is believed to play a role in mammalian development. Mice homozygous for a targeted mutation of the FX gene exhibit pleiotropic abnormalities including a lethal phenotype. Reduced recovery of mice from heterozygous crosses was also reported. (Becker et al., Mammalian Genome 14:130-139 (2003)). Aberrant protein fucosylation has been proposed to be associated with human disease, including up-regulation of sialyl Lewis^{X} in cancers. These glycans are ligands for E- and P-selectin molecules. It is speculated that increases in sialyl Lewis^{X} glycans on cancer cells increases metastases through interaction with E- and P-selectins on endothelium. In vivo administration of fucose analogs including 2-deoxy-2-fluoro-L-fucose, their effects on protein defucosylation, and their potential use for the treatment of cancer, have been described in International application WO 2012019165, which is herein incorporated by reference.

Immune checkpoints, which act as the off-switch on the T-cells of the immune system, have been investigated to reinstate the immune response with targeted agents, thus indirectly treating cancer by activating the body's immune system.

International applications WO2002086083, WO2004004771, WO2004056875, WO2006121168, WO2008156712, WO2010077634, WO2011066389, WO2014055897 and WO2014100079 report PD-1, PD-L1 inhibitory antibodies and/or methods of identifying such antibodies. Further, U.S. patents such as U.S. Pat. No. 8,735,553 and U.S. Pat. No. 8,168,757 report PD-1 or PD-L1 inhibitory antibodies and/or fusion proteins. The disclosures of WO2002086083, WO2004004771, WO2004056875, WO2006121168, WO2008156712, WO2010077634, WO2011066389, WO2014055897 and WO2014100079, as well as U.S. Pat. No. 8,735,553 and U.S. Pat. No. 8,168,757, are incorporated herein by reference in their entirety.

Moreover, International applications WO2011161699, WO2012168944, WO2013144704, WO2013132317 and WO 2016044900 report peptides or peptidomimetic compounds which are capable of suppressing and/or inhibiting the programmed cell death 1 (PD-1) signaling pathway. The disclosures of WO2011161699, WO2012168944, WO2013144704, WO2013132317 and WO 2016044900 are incorporated herein by reference in their entirety.

Furthermore, International applications WO 2016142852, WO 2016142894, WO 2016142886, WO 2016142835 and WO 2016142833 report small molecule compounds which are capable of suppressing and/or inhibiting the programmed cell death 1 (PD-1) signaling pathway and/or treating disorders by inhibiting an immunosuppressive signal induced by PD-1, PD-L1 or PD-L2. The disclosures of WO 2016142852, WO 2016142894, WO 2016142886, WO 2016142835 and WO 2016142833 are incorporated herein by reference in their entirety.

Recently, ipilimumab (Yervoy®), a monoclonal antibody that targets cytotoxic

T-lymphocyte-associated antigen 4 (CTLA-4) and nivolumab (Opdivo®), a monoclonal antibody that targets the programmed cell death protein 1 pathway (PD-1) on the surface of T-cells, have been approved by the U.S. Food and Drug Administration for the treatment of advanced melanoma, advanced renal cell carcinoma, and non-small cell lung cancer. Current checkpoint inhibitor therapies, however, are effective at treating cancer in a relatively small population of cancer subject population which is in part due to pre-existing immune activation and presence of the inhibitory receptors. Accordingly, there is a need to develop methods and combination therapies to initiate or enhance the effectiveness of the checkpoint inhibitors in both the nonresponding subject population and the responding subject population.

### SUMMARY OF THE INVENTION

The present invention discloses a combination treatment using 2-deoxy-2-fluoro-L-fucose (also referred to herein as “2FF”) and a checkpoint inhibitor, that are effective in treating cancer or inhibiting the proliferation of tumor cells in a subject and/or that can initiate, enhance or prolong the immune response to tumor cells.

### BRIEF DESCRIPTION OF THE DRAWINGS

FIG. 1A shows in vivo effects of 2-deoxy-2-fluoro-L-fucose on the growth of IV implanted A20 mouse lymphoma cells in BALB/c mice with or without vaccination with KLH-conjugated A20 Id Fab (20 mM 2-deoxy-2-fluoro-L-fucose in drinking water).

FIG. 1B shows effect of immune cell subset depletion on the in vivo effects of 2-deoxy-2-fluoro-L-fucose on the growth of IV implanted A20 mouse lymphoma cells in BALB/c mice in combination with vaccination with KLH-conjugated A20 Id Fab (20 mM 2-deoxy-2-fluoro-L-fucose in drinking water).

FIG. 2A shows measurement of dendritic cell (also referred to as “DC”) markers on DCs co-cultured with T-cells in serum-containing medium (MFI of 2-deoxy-2-fluoro-L-fucose-treated co-culture compared to control co-culture).

FIG. 2B shows measurement of DC markers on DCs co-cultured with T-cells in Serum-free medium (MFI of 2-deoxy-2-fluoro-L-fucose-treated serum-free co-culture compared to control serum-fee co-culture).

FIG. 3 shows comparison of tetramer binding to T-cells (MFI of binding to 2-deoxy-2-fluoro-L-fucose-treated T-cells compared to control T-cells) for three different tetramers (EBV, M1, and CMV).

FIG. 4 shows TCR-mediated phosphorylation of ZAP in CD3/CD28 activated T-cells (with or without 2-deoxy-2-fluoro-L-fucose treatment).

FIG. 5 shows expression of galectin-3 in T-cells with or without 2-deoxy-2-fluoro-L-fucose treatment evaluated by FACS.

FIG. 6 shows comparison of the phosphorylation of SMAD2 in 2-deoxy-2-fluoro-L-fucose-treated vs. control T-cells stimulated with TGFβ.

FIG. 7A and 7B show comparison of the FOXp3 expression and percentage of regulatory T-cells in 2-deoxy-2-fluoro-L-fucose-treated vs. control T-cells.

FIGS. 8A, 8B and 8C show T-cells/DC co-culture cytokines in (A) INFγ, (B) IL12p40, and (C) CD40L, respectively.

FIG. 9 shows A20 vaccine model cytokine evaluation, KLH-Idiotype (control) vs. 2-deoxy-2-fluoro-L-fucose+KLH-Idiotype (2-deoxy-2-fluoro-L-fucose) (BALB/c mice).

FIGS. 10A and 10B show in vitro production of IFNγ and IL-12p70, respectively, in human co-cultures.

FIG. 11 shows in vivo effects of 2-deoxy-2-fluoro-L-fucose, anti-PD1 antibody and the combination of 2FF with anti-PD1 antibody on the growth of IV implanted A20 mouse lymphoma cells in BALB/c mice (20 mM 2FF in drinking water, 5 mg/kg anti-PD1 q3x3).

FIGS. 12A-C show active immune response at the tumor microenvironment.

### DETAILED DESCRIPTION OF THE INVENTION

The present invention describes a novel combination treatment based on activating or enhancing the adaptive immune response. The adaptive immune response mechanism implies that a checkpoint inhibitor will only work or work more effectively when there is a pre-existing anti-tumor immune response (i.e., activated T-cells). For example, in patients who do not have preexisting anti-tumor responses, the checkpoint inhibitors alone will potentially not be effective. Accordingly, a combination treatment that can activate anti-tumor activity (e.g., an anti-tumor immune response) and inhibit the checkpoint blockade is preferable because it would allow subjects who do not respond to treatment by checkpoint inhibitors alone to benefit from this combined treatment.

The present invention provides, inter alia, methods for treating cancers, comprising administering to a subject in need thereof an effective amount of 2-deoxy-2-fluoro-L-fucose or a prodrug thereof, or a pharmaceutically acceptable salt thereof, in combination with a checkpoint inhibitor. The present invention also provides, inter alia, methods for inhibiting the proliferation of a tumor in a subject in need thereof comprising administering to the subject an effective amount of 2-deoxy-2-fluoro-L-fucose or a prodrug thereof, or a pharmaceutically acceptable salt thereof, in combination with a checkpoint inhibitor. The present invention further provides, inter alia, methods for enhancing or prolonging the effects of a checkpoint inhibitor, or enabling a subject to respond to a checkpoint inhibitor in a subject in need thereof comprising administering to the subject an effective amount of 2-deoxy-2-fluoro-L-fucose or a prodrug thereof, or a pharmaceutically acceptable salt thereof, in combination with a checkpoint inhibitor.

In some aspects, the combination of 2-deoxy-2-fluoro-L-fucose or a prodrug thereof, or a pharmaceutically acceptable salt thereof, and the checkpoint inhibitor administered provides an additive or synergistic effect in the treatment of the cancer or in the inhibition of the proliferation of tumor cells. In another aspect, the combination of 2-deoxy-2-fluoro-L-fucose or a prodrug thereof, or a pharmaceutically acceptable salt thereof, and the checkpoint inhibitor administered provides a synergistic effect in the treatment of the cancer or in the inhibition of the proliferation of tumor cells. In another aspect, 2-deoxy-2-fluoro-L-fucose or a pharmaceutically acceptable salt thereof is administered in combination with a checkpoint inhibitor. In another aspect, the prodrug of 2-deoxy-2-fluoro-L-fucose or a pharmaceutically acceptable salt thereof is administered in combination with a checkpoint inhibitor. In some embodiments, carboxylic ester prodrug of 2-deoxy-2-fluoro-L-fucose or a pharmaceutically acceptable salt thereof is administered in combination with a checkpoint inhibitor. In some embodiments, an acetate ester of 2-deoxy-2-fluoro-L-fucose or a pharmaceutically acceptable salt thereof is administered in combination with a checkpoint inhibitor.

In some embodiments, the checkpoint inhibitor is a biologic therapeutic or a small molecule. In some embodiments, the checkpoint inhibitor is selected from the group consisting of a monoclonal antibody, a humanized antibody, a fully human antibody and a fusion protein or a combination thereof. In some embodiments, the checkpoint inhibitor inhibits or interact with a ligand of a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN -15049, CHK 1, CHK2, A2aR, and B-7 family ligands or a combination thereof. In some embodiments, the checkpoint inhibitor is a PD-L1, PD-L2, or PD-1 inhibitor. In some embodiments, the checkpoint inhibitor is a PD-1 inhibitor. In some embodiments, the checkpoint inhibitor is a PD-1 inhibitor is nivolumab or pembrolizumab. In another embodiment, the checkpoint inhibitor is a PD-1 inhibitor is nivolumab.

In some embodiments, the cancer is a solid tumor. In some embodiments, the cancer is a liquid tumor. In some embodiments, the cancer is selected from the group consisting of urogenital, gynecological, lung, gastrointestinal, head and neck cancer, brain cancers including malignant gliomas and brain metastases, malignant mesothelioma, non-metastatic or metastatic breast cancer, malignant melanoma, Merkel Cell Carcinoma or bone and soft tissue sarcomas, haematologic neoplasias, multiple myeloma, lymphomas such as Hodgkin's disease, non-Hodgkin's lymphoma, acute myelogenous leukemia, chronic myelogenous leukemia, myelodysplastic syndrome and acute lymphoblastic leukemia, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer. In some embodiments, the cancer is non-small cell lung cancer (NSCLC), breast cancer, or a colorectal cancer. In some embodiments, the cancer is non-small cell lung cancer (NSCLC). In some embodiments, a composition comprising 2-deoxy-2-fluoro-L-fucose or a prodrug thereof, or a pharmaceutically acceptable salt thereof, is administered to the subject. In some embodiments, the composition is a solid or a liquid formulation. In some embodiments, the checkpoint inhibitor and 2-deoxy-2-fluoro-L-fucose are administered simultaneously or sequentially, in either order. In some embodiments, the subject is a mammal. In some embodiments, the mammal is a human.

### Definitions

The terms “inhibit” or “inhibition of” means to reduce by a measurable amount, or to prevent entirely. The term inhibition as used herein can refer to an inhibition or reduction of at least about 10%, at least about 15%, at least about 20%, at least about 25%, at least about 30%, at least about 35%, at least about 40%, at least about 50%, at least about 60%, at least about 70%, at least about 80%, at least about 90%, at least about 95%, or at least about 99%.

The terms “treatment” or “treat” refer to slowing, stopping, or reversing the progression of the disease or condition in a patient, as evidenced by a decrease or elimination of a clinical or diagnostic symptom of the disease or condition. Treatment can include, for example, a decrease in the severity of a symptom, the number of symptoms, or frequency of relapse.

The term “pharmaceutically acceptable” means approved by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which 2-deoxy-2-fluoro-L-fucose is administered.

The term “prodrug”, as used herein, refers to a compound that is converted into the active form of the compound upon administration in vivo. For example, a prodrug form of an active compound can be, but not limited to, acylated (acetylated or other) and ether derivatives, carboxylic esters or phosphate esters and various salt forms of the active compound. One of ordinary skill in the art will recognize how to readily modify the compound of subject invention to a prodrug form to facilitate delivery of active compound to a targeted site within the host organism or patient. The skilled artisan also will take advantage of favorable pharmacokinetic parameters of the prodrug form, where applicable, in delivering the desired compound to a targeted site within the host organism or patient to maximize the intended effect of the compound in the treatment of cancer.

2-deoxy-2-fluoro-L-fucose or a prodrug thereof, or a pharmaceutically acceptable salt thereof, is typically substantially pure from undesired contaminant. This means that 2-deoxy-2-fluoro-L-fucose or a prodrug thereof, or a pharmaceutically acceptable salt thereof, is typically at least about 50% w/w (weight/weight) or about 80% w/w purity, more preferably at least about 90% or about 95% w/w purity, as well as being substantially free from impurities and other contaminants. Using conventional purification techniques, homogeneous product of at least 99% w/w can be obtained.

The term “subject” or “patient” for purposes of treatment refers to any animal, particularly an animal classified as a mammal, including humans, domesticated and farm animals, and zoo, sports, or pet animals, such as dogs, horses, cats, cows, and the like. Preferably, the subject is human.

The term “therapeutically effective amount” or “effective amount” refers to the amount of one or more agents or compositions as described herein that is sufficient to slow, stop, or reverse the progression of cancer in a subject or increase survival of the patient. The therapeutically effective amount may refer to a target serum concentration that has been shown to be effective in, for example, slowing disease progression. When the term “therapeutically effective amount” is used to refer to combination therapy, it refers to the amount of the combination of agents taken together so that the combined effect elicits the desired biological or medicinal response. Efficacy can be measured in conventional ways, depending on the condition to be treated. For example, in neoplastic diseases, efficacy can be measured by assessing the time to disease progression (TTP), or determining the response rates (RR).

As used herein, the term “synergy” or “synergistic effect” when used in connection with a description of the efficacy of a combination of agents, means any measured effect of the combination which is greater than the effect predicted from a sum of the effects of the individual agents.

As used herein, the term “additive” or “additive effect” when used in connection with a description of the efficacy of a combination of agents, means any measured effect of the combination which is similar to the effect predicted from a sum of the effects of the individual agents.

### Therapeutic Methods

2-deoxy-2-fluoro-L-fucose in solid phase has a chemical structure (I) as the following:

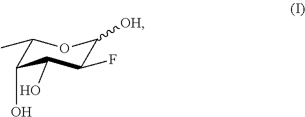

which can be the alpha or beta anomer.

In solution, 2-deoxy-2-fluoro-L-fucose can interconvert via the corresponding aldose form to a compound having a chemical structure (II) as the following:

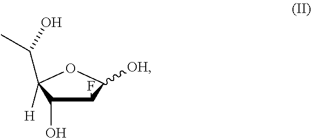

Accordingly, the term “2-deoxy-2-fluoro-L-fucose”, as used herein, refers to a compound of formula (I), (II) or the corresponding aldose form, or a mixture thereof, wherein each of formula (I) or (II) can independently be alpha or beta anomer.

2-deoxy-2-fluoro-L-fucose is described in WO 2009/135181 that reduces the incorporation of fucose into complex N-glycoside-linked sugar chains of antibodies or antibody derivatives produced by host cells in vitro. 2-deoxy-2-fluoro-L-fucose, as well as its potential anti-tumor effects, is described in WO 2012/019165 that inhibits protein fucosylation when administered in vivo. The disclosures of both WO 2009/135181 and WO 2012/019165 are incorporated by reference in their entirety in the disclosure of this application.

It has been surprisingly found that 2-deoxy-2-fluoro-L-fucose reduces regulatory T-cells. In the methods provided herein 2-deoxy-2-fluoro-L-fucoses can be used as an immuno-modulatory agent to treat cancer due to the changes in T-cell activity induced when they are afucosylated by treatment with 2-deoxy-2-fluoro-L-fucose. Changes that occur include reduction in regulatory T-cell populations, increases in T-cell activation of antigen presenting cells, as well as increased T-cell receptor signaling which can in turn result in the increased activation of APCs. The result of this modulation of T-cell activity would be to reduce the immunosuppressive tumor microenvironment as well as increasing T-cell and APC activation. Thus, treatment with 2-deoxy-2-fluoro-L-fucose would lead to an increased host-mediated anti-tumor immune response resulting in delay of tumor progression or delay to tumor onset. The anti-tumor activity may also occur with direct, adoptive transfer of 2-deoxy-2-fluoro-L-fucoses treated T-cells. 2-deoxy-2-fluoro-L-fucose for the methods provided herein can be safely administered in combination with a checkpoint inhibitor to a subject in an amount effective to treat cancer in the subject, such as a human in need thereof. In the methods provided herein, 2-deoxy-2-fluoro-L-fucose in combination with a checkpoint inhibitor also provides a better safety profile than the existing PD-1 combination treatments such as the combination of a PD-1 inhibitor and a CTLA-4 inhibitor.

In some embodiments, 2-deoxy-2-fluoro-L-fucose (or an intracellular metabolite or product of 2-deoxy-2-fluoro-L-fucose) increases humoral and cellular immune responses when administered with a cancer vaccine. In some embodiments, 2-deoxy-2-fluoro-L-fucose (or an intracellular metabolite or product of 2-deoxy-2-fluoro-L-fucose) increases CD45RO1+ T-cell population (memory T-cell phenotype). In some embodiments, 2-deoxy-2-fluoro-L-fucose (or an intracellular metabolite or product of 2-deoxy-2-fluoro-L-fucose) treated T-cells activate dendritic cells more than non-treated (control) T-cells. In some embodiments, 2-deoxy-2-fluoro-L-fucose (or an intracellular metabolite or product of 2-deoxy-2-fluoro-L-fucose) increases antigen-specific (for example, EBV-specific) tetramer binding. In some embodiments, 2-deoxy-2-fluoro-L-fucose (or an intracellular metabolite or product of 2-deoxy-2-fluoro-L-fucose) decrease regulatory T-cell populations, as well as the number of FOXp3+ cells. Anti-CTLA4 checkpoint inhibitor has been shown to decrease regulatory T-cell populations and has been successfully combined with other checkpoint inhibitors with different mechanistic targets such as anti-PD1 and anti-PD-L1 antibodies. Thus, in some embodiments, 2-deoxy-2-fluoro-L-fucose (or an intracellular metabolite or product of 2-deoxy-2-fluoro-L-fucose) is administered in combination with another checkpoint inhibitors such as an anti-PD1 or anti-PD-L1 antibody.

2-deoxy-2-fluoro-L-fucose provided herein is useful for treating cancer in a subject. Administration of 2-deoxy-2-fluoro-L-fucose to an animal (e.g., a mammal, such as a human) in need thereof can result in inhibition of the multiplication of a tumor cell(s) or cancer cell(s), or treatment of cancer in an animal (e.g., a human patient). 2-deoxy-2-fluoro-L-fucose can be used accordingly in a variety of settings for the treatment of animal cancers.

Particular types of cancers that can be treated with 2-deoxy-2-fluoro-L-fucose include, solid tumors and hematologic malignancies. Such cancers include, but are not limited to: (1) solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias, and (3) lymphomas such as Hodgkin's disease, non-Hodgkin's Lymphoma, multiple myeloma, Waldenstrom's macroglobulinemia, Heavy chain disease, and Polycythemia vera.

In some aspects, 2-deoxy-2-fluoro-L-fucose is soluble in formulation buffer (e.g. aqueous formulation buffer) at a concentration of at least 10 mM. In some embodiments, 2-deoxy-2-fluoro-L-fucose is soluble in formulation buffer at a concentration of at least 100 mM. In some aspects, 2-deoxy-2-fluoro-L-fucose is soluble in formulation buffer (e.g. aqueous formulation buffer) at a concentration of at least 100 μg/ml, at least 1 mg/ml, at least 50 mg/ml, at least about 100 mg/ml, at least about 200 mg/ml, or at least about 300 mg/ml.

2-deoxy-2-fluoro-L-fucose or its prodrug, or a pharmaceutically acceptable salt thereof, can be formulated as pharmaceutical compositions comprising a therapeutically or prophylactically effective amount of 2-deoxy-2-fluoro-L-fucose or its prodrug, or a pharmaceutically acceptable salt thereof and one or more pharmaceutically compatible (acceptable) ingredients. In some aspects, pharmaceutical compositions of 2-deoxy-2-fluoro-L-fucose and pharmaceutical excipients are provided in which an effective amount of 2-deoxy-2-fluoro-L-fucose(s) is in admixture with the excipients, suitable for administration to a mammal In preferred aspects, 2-deoxy-2-fluoro-L-fucose is formulated for administration to a human. According, the present invention provides a pharmaceutical composition comprising 2-deoxy-2-fluoro-L-fucose formulated for administration to a human. The formulated 2-deoxy-2-fluoro-L-fucose will generally comprise one or more pharmaceutically compatible (acceptable) ingredients.

Exemplary pharmaceutical or non-pharmaceutical compositions typically include one or more carriers (e.g., sterile liquids, such as water and oils, including those of petroleum, animal, vegetable or synthetic origin, such as peanut oil, soybean oil, mineral oil, sesame oil and the like). Water is a more typical carrier when the pharmaceutical composition is administered intravenously. Saline solutions and aqueous dextrose and glycerol solutions can also be employed as liquid carriers, particularly for injectable solutions. Suitable excipients include, for example, amino acids, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin. Such compositions will typically contain a therapeutically effective amount of 2-deoxy-2-fluoro-L-fucose, typically in purified form, together with a suitable amount of carrier so as to provide the form for proper administration to the subject. The formulations correspond to the mode of administration.

The pharmaceutical compositions described herein can be in any form that allows for the composition to be administered to an animal (e.g., a mammal). The compositions can be in the form of a solid or liquid. Typical routes of administration include, without limitation, oral, parenteral, and sublingual. Parenteral administration includes subcutaneous injections, intraperitoneal injections, intravenous, intramuscular, intrasternal injection or infusion techniques. Preferably, the compositions are administered orally. These pharmaceutical compositions can be formulated so as to allow 2-deoxy-2-fluoro-L-fucose to be bioavailable upon administration of the composition to an animal. Compositions can also take the form of one or more dosage units, where for example, a tablet can be a single dosage unit, and a container of 2-deoxy-2-fluoro-L-fucose in solid form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of 2-deoxy-2-fluoro-L-fucose, the manner of administration, the composition employed, and the severity of the disease or condition being treated.

The pharmaceutically acceptable carrier or vehicle can be particulate, so that the compositions are, for example, in tablet or powder form. The carrier(s) can be liquid, with the compositions being, for example, an oral syrup, flavored water, or injectable liquid.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it can contain, in addition to materials of the above type, a liquid carrier such as polyethylene glycol, cyclodextrin or fatty oil.

The composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant, and flavor enhancer. In some aspects, the composition is formulated into a powder and the end user mixes the power in an aqueous solution for oral administration. In a composition for administration by injection (as described above), one or more of a surfactant, preservative, wetting agent, dispersing agent, suspending agent, buffer, stabilizer and isotonic agent can also be included.

As noted above, the amount of 2-deoxy-2-fluoro-L-fucose that is effective in the methods described herein will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of 2-deoxy-2-fluoro-L-fucose such that a suitable dosage will be obtained. Typically, this amount is at least about 0.01% of 2-deoxy-2-fluoro-L-fucose by weight of the composition. In some aspects, when intended for oral administration, this amount can be varied to range from about 0.1% to about 100% by weight of the composition. Preferred oral compositions can comprise, for example, from about 4% to 100%, 4% to 75% or from 4% to about 50% of 2-deoxy-2-fluoro-L-fucose by weight of the composition.

In some aspects, for intravenous administration, the amount administered will be in the range from about 1 to about 500 mg/kg of body weight of 2-deoxy-2-fluoro-L-fucose.

Generally, the oral dosage of 2-deoxy-2-fluoro-L-fucose administered to an animal is about 1 mg/kg to about 1 g/kg of the animal's body weight, more typically about 5 mg/kg, 10 mg/kg, 25 mg/kg, 50 mg/kg, 100 mg/kg, 150 mg/kg, 200 mg/kg, 250 mg/kg, or 300 mg/kg to about 500 mg/kg of the animal's body weight. In some aspects, the dosage administered to an animal is about 1 g, about 5 g, or about 10 g to about 150 g per day, or from about 1 g, about 5 g, about 10 g, about 15 g or about 20 g to about 60 g per day.

Generally, 2-deoxy-2-fluoro-L-fucose or a pharmaceutical composition thereof can be administered on a daily, weekly, biweekly or monthly schedule, according to the desired effect. In some aspects, 2-deoxy-2-fluoro-L-fucose or a pharmaceutical composition thereof can be administered from about 1 to 5, about 1 to about 10, about 1 to about 15, or more cycles, wherein each cycle is a month in duration. The doses within each cycle can be given on daily (including once daily, twice daily, or more than twice daily), every other day, twice weekly, weekly, bi-weekly, once every three weeks or monthly basis. A cycle may optionally include a resting period. Alternatively, a resting period can be included between cycles. In some aspects, administration will be for the duration of the disease.

The preferred mode of administration of 2-deoxy-2-fluoro-L-fucose, or a pharmaceutical composition thereof, is left to the discretion of the practitioner, and will depend in-part upon the site of the medical condition. In one embodiment, 2-deoxy-2-fluoro-L-fucose or compositions are administered parenterally. In another embodiment, 2-deoxy-2-fluoro-L-fucose or compositions are administered orally.

In another embodiment, 2-deoxy-2-fluoro-L-fucose can be delivered in a vesicle, in particular a liposome (see Langer, Science 249:1527-1533 (1990); Treat et al., in LIPOSOMES IN THE THERAPY OF INFECTIOUS DISEASE AND CANCER, Lopez-Berestein and Fidler (eds.), Liss, N.Y., pp. 353-365 (1989); Lopez-Berestein, ibid., pp. 317-327; see generally ibid.).

In yet another embodiment, 2-deoxy-2-fluoro-L-fucose or compositions can be delivered in a controlled release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see MEDICAL APPLICATIONS OF CONTROLLED RELEASE, Langer and Wise (eds.), CRC Pres., Boca Raton, Fla. (1974); CONTROLLED DRUG BIOAVAILABILITY, DRUG PRODUCT DESIGN AND PERFORMANCE, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351(1989); Howard et al., J. Neurosurg. 71:105 (1989)). Other controlled-release systems discussed in the review by Langer (Science 249:1527-1533 (1990)) can be used.

The term “carrier” refers to a diluent, adjuvant or excipient, with which 2-deoxy-2-fluoro-L-fucose is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, 2-deoxy-2-fluoro-L-fucose or compositions and pharmaceutically acceptable carriers are sterile. Water is a preferred carrier when 2-deoxy-2-fluoro-L-fucose are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response (‘block’ the immune response) against tumor tissues. In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264) Therapy with antagonistic checkpoint blocking antibodies against immune system checkpoints such as CTLA4, PD1 and PD-L1 are one of the most promising new avenues of immunotherapy for cancer and other diseases. Additional checkpoint targets, such as TIM-3, LAG-3, various B-7 ligands, CHK 1 and CHK2 kinases, BTLA, A2aR, and others, are also under investigation. Currently, three checkpoint inhibitors have received rapid approval from the U.S. Food and Drug Administration for cancer treatment, including ipilimumab (Yervoy®), a CTLA-4 inhibitor, and pembrolizumab (Keytruda®) and nivolumab (Opdivo®), both PD-1 inhibitors. In addition, several checkpoint inhibitor agents are in clinical trials.

Recent data suggest a secondary mechanism of anti-CTLA-4 antibodies, which could occur within the tumor itself. CTLA-4 has been found to be expressed in tumors at higher levels on regulatory T-cells (also referred to herein as “Treg cells”) as compared with intra-tumoral effector T-cells (also referred to herein as “Teff cells”), resulting in the hypothesis of anti-CTLA-4 preferentially impacting the Treg cell. “Therapeutic use of anti-CTLA-4 antibodies”, Christian U. Blank and Alexander Enk, International Immunology, Vol. 27, No. 1, pp. 3-10. A recent study of a PD-1 and CTLA-4 combination show that the combination blockade of the CTLA-4 and PD-1 pathways also cooperates to increase the ratio of Teff cells to both regulatory T-cells and MDSCs, thereby reducing suppression and promoting inflammation in the tumor microenvironment. “Combination of CTLA-4 and PD-1 blockade expands infiltrating T-cells and reduces regulatory T and myeloid cells within B16 melanoma tumors”, Curran et al., PNAS|Mar. 2, 2010|vol. 107|no. 9|4275-4280. The combination of a checkpoint inhibitor and another therapeutic agent(s) may enhance or prolong an anti-tumor response of the checkpoint inhibitor and/or effects of the therapeutic agent. In this regard, WO 2015/069770 discloses a combination treatment based on activating the adaptive immune response, in particular the combination of CTLA-4 and PD-1 inhibitors, for the treatment of cancer. The disclosure of WO 2015/069770 is incorporated by reference in its entirety in the disclosure of this application.

One mechanism by which the checkpoint blockade anti-CTLA-4 antibodies mediate anti-tumor effect is by decreasing regulatory T-cells. Due to the distinct mechanism of action of anti-CTLA-4 antibodies, they can successfully combine with the anti-PD1 checkpoint blockade antibodies which work to release the suppressive signaling conferred to effector T-cells. Dual blockade with these antibodies combine to improve anti-tumor response both preclinically (Proc Natl Acad Sci USA 2010, 107, 4275-4280) and in the clinic (N Engl J Med 2013, 369, 122-133; N Engl J Med 2015, 372, 2006-2017).

Generally, the optimal amount of 2-deoxy-2-fluoro-L-fucose and the checkpoint inhibitor that is effective in the treatment of cancer can be determined by standard clinical techniques. In addition, in vitro assays may optionally be employed to help identify optimal dosage ranges. The precise dose to be employed in the formulation will also depend on the route of administration, and the stage of malignancy, and should be decided according to the judgment of the practitioner and each patient's circumstances. Effective doses may be extrapolated from dose-response curves derived from in vitro or animal model test systems.

In a further aspect, the checkpoint inhibitor and 2-deoxy-2-fluoro-L-fucose are administered simultaneously or sequentially, in either order. In a specific aspect, the checkpoint inhibitor is a PD-1 inhibitor or CTLA-4 inhibitor. In another specific aspect, the checkpoint inhibitor is a PD-1 inhibitor.

In some embodiments, 2-deoxy-2-fluoro-L-fucose and the checkpoint inhibitor will be administered to a subject at the Maximal Tolerable Dose (MTD) or the Optimal Biological Dose (OBD). It is within the art to determine MTD or OBD. In some aspects, 2-deoxy-2-fluoro-L-fucose will be provided at its MTD or OBD and the checkpoint inhibitor will be dosed at 50%-100%, preferably at 50% to 90% of the MTD or OBD. Alternatively, the checkpoint inhibitor will be dosed at its MTD or OBD and 2-deoxy-2-fluoro-L-fucose will be dosed at at 50%-100%, preferably at 50% to 90% of the MTD or OBD. In some aspects, both 2-deoxy-2-fluoro-L-fucose and the checkpoint inhibitor will be dosed at 60% to 90% of the MTD or OBD.

As used in this invention, the combination regimen can be given simultaneously or can be given in a staggered regimen, with the checkpoint inhibitor being given at a different time during the course of therapy than 2-deoxy-2-fluoro-L-fucose. This time differential may range from several minutes, hours, days, weeks, or longer between administration of the two agents. Therefore, the term combination does not necessarily mean administered at the same time or as a unitary dose, but that each of the components are administered during a desired treatment period. The agents may also be administered by different routes.

Also provided herein is the prodrug of 2-deoxy-2-fluoro-L-fucose or a pharmaceutically acceptable salt of 2-deoxy-2-fluoro-L-fucose or its prodrug. Accordingly, in any of the various embodiments provided herein, the prodrug of 2-deoxy-2-fluoro-L-fucose or a pharmaceutically acceptable salt of 2-deoxy-2-fluoro-L-fucose or its prodrug can be used.

These and other aspects of the present invention may be more fully understood by reference to the following detailed description, non-limiting examples of specific embodiments, and the appended figures.

### EXAMPLES

Examples are provided to assist in a further understanding of the inventions. Particular materials used, protocols and conditions are intended to be further illustrative of the inventions and should not be construed to limit the reasonable scope thereof.

### Example 1

### A20 Mouse Lymphoma Study with Immune Subset Depletion

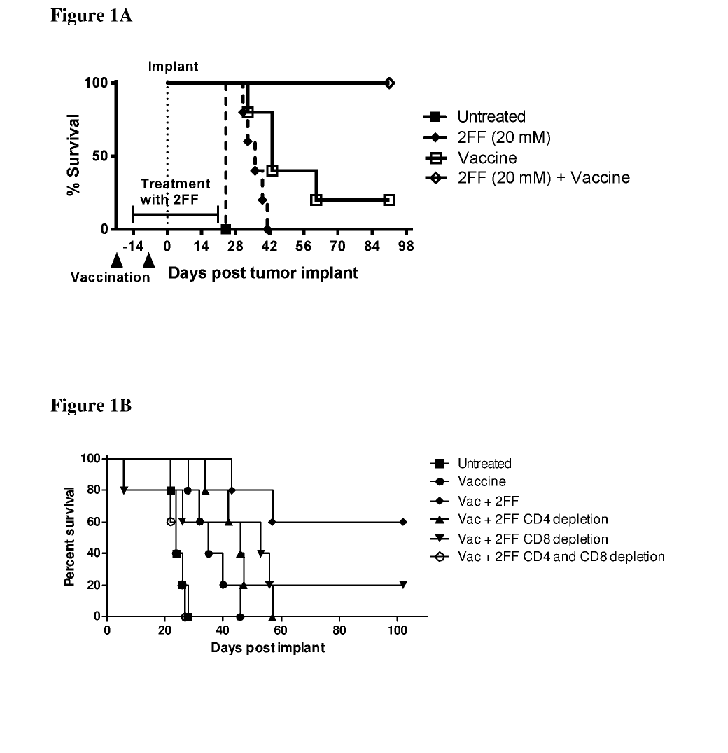

KLH-A20 Id Fab was generated as described in Okeley et al PNAS 2012. A20 cells (ATCC) were cultured in RPMI 1640 with 10% FBS, 10 mM HEPES, 1 mM sodium pyruvate, 50 μM 2-mercaptoethanol and penicillin (100 U/ml)/streptomycin (100 μg/ml) (PS) Immunization groups (BALB/c, Harlan) were injected subcutaneously with the KLH-Fab conjugate (50 μg) with TiterMax adjuvant (1:1) on day-21 with a boost on day-7. 2-deoxy-2-fluoro-L-fucose treatment groups received drinking water containing 20 mM 2-deoxy-2-fluoro-L-fucose beginning on day -14. One week after the second vaccination (day 0), all mice received 2.5×10⁶ A20 tumor cells (i.v.). 2-deoxy-2-fluoro-L-fucose treatment was continued until day 21 followed by normal drinking water. Depletion of immune cells was accomplished with depleting antibodies administered on days-6, -5, -4, 0 and +7 (200 μg/mouse i.p.). CD4 or CD8 T-cells depletion by anti-CD4 (GK1.5) or -CD8 (53-6.72) antibody was verified in blood at day-1, 7, 14, 21, 29 (FACS analysis). Similar results were seen in spleen at day 0 and 29 from BALB/c mice. FIGS. 1A and 1B demonstrate that depletion of CD4 or CD8 T-cells reduces the activity of the A20 idiotype vaccine plus 2-deoxy-2-fluoro-L-fucose combination. Thus CD4 and CD8 T-cells play a role in the combination activity.

### Example 2

### T-Cell and Dendritic Cell Isolation from Whole Blood

T-cells were isolated from 10 mL of whole blood which was centrifuged first at 1200 rpm (300×g) for 10 min (without brakes). The top layer containing platelets was removed carefully without disrupting the white blood cell layer. Then RosetteSep™ Human T-Cell Enrichment Cocktail (Pan T-cells from StemCell technologies, Vancouver, BC) was added to the remaining blood (500 μL/10 mL blood). This was incubated for 20 min and then 1 mL FBS was added along with 10 mL PBS. Histopaque (20-25 mL) placed in a 50 mL Falcon the prepared blood/PBS solution was overlaid very slowly. This was centrifuged with no brake (25° C., 1500 rpm, 25 min). The top layer was removed and the T-cells in the buffy coat layer were then removed to a new 50 mL tube. The T-cells were washed with PBS, resuspended in 1 mL ACK lysis buffer and topped up to 25 mL, incubated for 5 min, brought up to 50 mL with PBS, and then pelleted. This red blood cell lysis step was repeated a second time. The T-cells were then resuspended in T-cell media (RPMI media supplemented with 10% Fetal calf serum (FCS), 1% PS and spilt into two T25 flasks (with or without 100-200 μM 2-deoxy-2-fluoro-L-fucose). CD3/CD28 antibody coated beads (20 μL/flask, Miltenyi Biotec) were added to activate the T-cells. After 24 hr IL2 was added (100 ng/μL, R&D Systems). Each time the cells are passaged new IL2 and 2-deoxy-2-fluoro-L-fucose were added.

PBMCs were isolated similarly to the T-cells, except that 90 mL of blood were used and after the removal of platelets the RosetteSep™ Human T-Cell Enrichment Cocktail was not added, instead an equal volume of PBS (90 mL) was added before overlaying on Histopaque. Centrifugation over Histopaque as above results in PBMC-containing buffy coat. After red blood cell lysis, the PBMCs were resuspended in dendritic cell (DC) media (30% DMEM 70% X-VIVO™ 15+2 mM glutamax+10% ATB serum+1% PS), were plated into a 6-well plate and incubated overnight. The following day the supernatant was aspirated and discarded while the adhering cells were washed in media and then replenished with 2 mL media supplemented with IL4 (100 ng/μL, R&D Systems) and GM-CSF (200 ng/μL, R&D Systems) per well to differentiate and proliferate the adhering monocytes into DCs. The plate was incubated for 4-5 days and then cells moved into a T25 flask. Each time the cells were passaged (every 2-3 days) new cytokines were given.

### Co-Cultures of T-Cells and DCs

Co-culture experiments were performed in DC media at a ratio of 10:1 (T-cells:DCs) in 48 well plates. DCs (20000-30000/well) and 10 times as many T-cells were plated in each well in a total of 200 μL/well in DC medium. Co-cultures were incubated 24 h after which cells evaluated for DC maturation markers as described below.

### Transwell Co-Culture

Transwell assays were performed in 24-well plates. Co-cultures were plated out as described above with the final volume per well of 500 μL. For the transwell samples DCs were plated in the well and T-cells placed into the insert. The cells were then incubated 24 h and examined in the same manner as co-cultures without transwells.

### Serum Free Co-Culture

DCs were grown as described until day 8 when there were split into two flasks, one with normal media and the other in serum free media. While no serum was present, X-VIVO™ 15 media contains growth factors which help maintain the health of the cultures. The co-culture experiment was performed as above in 48-well plates.

### Analysis of DC Markers in Co-Cultures

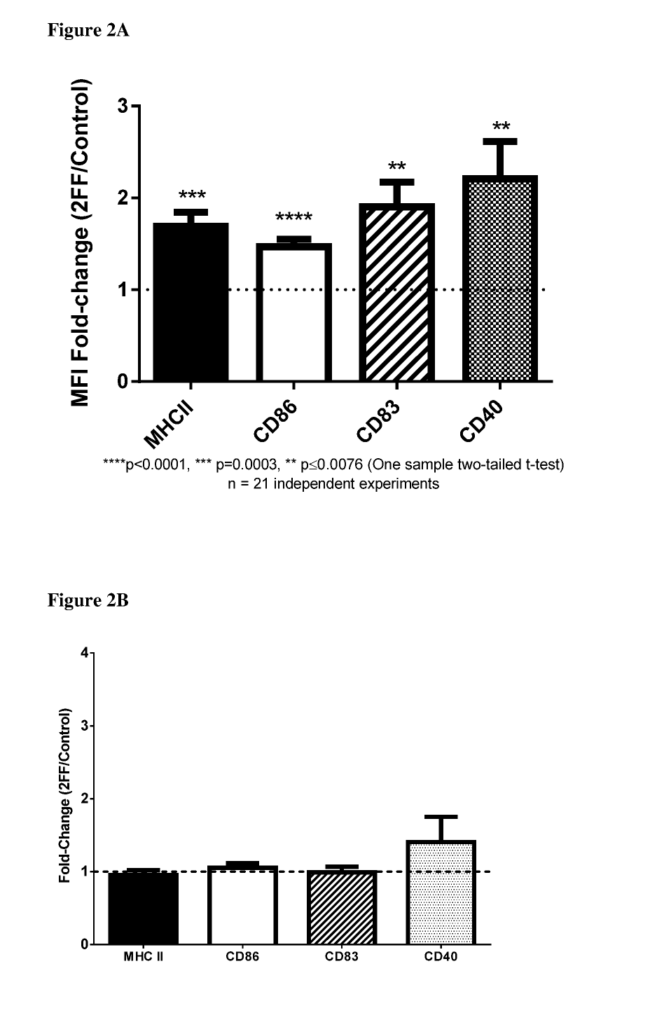

Co-cultures were examined using FACS analysis. After incubation, cells were washed in BD stain buffer (BSA) with Human Fc block (EMD Millipore) and incubated on ice for 30 min. Cells were then stained with fluorescently labeled anti-MHCII, anti-CD86, anti-CD83, and anti-CD40 primary antibodies (BD, 1:100 or 1:50 in BD stain buffer (BSA)) or with appropriate isotype controls (ice, dark, 40 min). Cells were then washed twice in BD stain buffer (BSA) and analyzed on an LSRII flow cytometer. DCs were identified by forward and side scatter and the MFI of each marker of interest was compared in 2-deoxy-2-fluoro-L-fucose-treated cultures to that of the control cultures. FIGS. 2A and 2B demonstrate that the fold difference between these markers for both serum-containing and serum-free assay conditions.

FIG. 2B demonstrates that co-culture experiments of T-cells with autologous DCs revealed that 2-deoxy-2-fluoro-L-fucose-treated T-cells activate DCs more so than control T-cells. This is shown by increases in the DC activation and maturation markers MHCII, CD86, CD83 and CD40. These increases are contact dependent, since they do not occur when transwell inserts are used to separate T-cells and DCs, suggesting that a soluble factor is not likely to be solely responsible. DCs alone do not show changes in these markers either with or without 2-deoxy-2-fluoro-L-fucose treatment. This interaction also requires antigen to be present for the increases in these cell surface activation markers since serum-free medium does not provide the same increases.

### Example 3

### Tetramer Staining of T-Cells

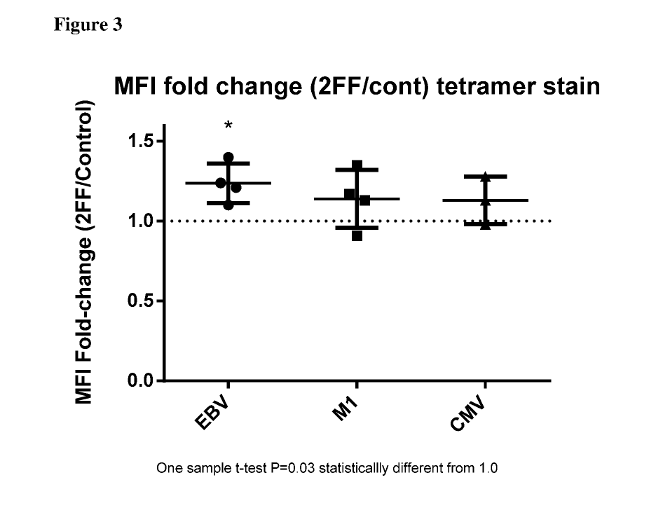

Antigen specific tetramers and negative control tetramer were purchased from MBL (Woburn, Mass.). T-cells expanded as described above with or without 2-deoxy-2-fluoro-L-fucose (100 μM) were plated in a round bottom 96-well plate, were centrifuged, and re-suspended in BD stain buffer (BSA) with Human Fc block (EMD Millipore) and incubated at RT for 10 min. Cells were then stained with the desired tetramer 10 μL/well or negative control and incubated for 30 min RT in the dark. Cells were centrifuged and washed in BD stain buffer (BSA) and then resuspened in cold BD BSA stain buffer for analysis with an LSRII flow cytometer. FIG. 3 demonstrates MFI fold change of binding to 2-deoxy-2-fluoro-L-fucose-treated T-cells compared to control T-cells for three different tetramers (EBV, M1, and CMV; MBL Bio).

### Example 4

### TCR Signaling

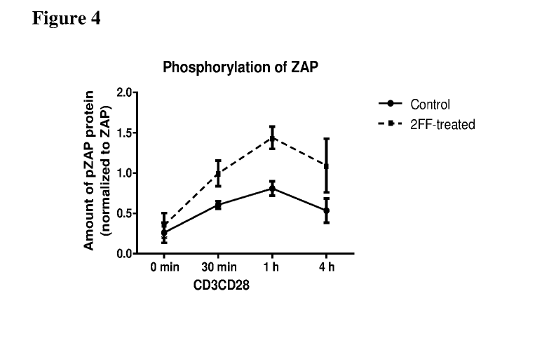

Purified T-cells were plated in 6-well plates (2.5×10⁶ cells/well, 2.5 mL T-cell medium) and were activated with CD3/CD28 beads (20 μL beads/well, Miltenyi Biotec, San Diego, Calif.) at 37 ° C. for 0-4 h. At the indicated time, samples were then harvested, washed in PBS, lysed with RIPA buffer (Thermo Scientific, containing DNAse and protease/phosphatase inhibitors), and snap frozen on dry ice followed by storage at −80 ° C. Cell lysates (˜3 μg/sample determined by BCA assay) were run on SDS-PAGE and examined by western blot on nitrocellulose membranes with anti-pZAP70 antibody (Cell Signaling Technologies). Briefly, blots were blocked for 1 h at room temperature in 5% milk in TBST (TBST from Cell Signling Technologies), rinsed with TBST and then incubated with primary antibody (anti-pZAP70 1:1000 in 5% BSA in TBST) at 4 ° C. overnight. Blots were washed 3× with TBST and probed with HRP-conjugated secondary antibodies for 1 hr at RT (1:2000 in 5% non-fat milk in TBST) followed by washing (3×TBST) and detection using Cell Signaling Elite ECL as per manufacturer instructions. Blots were scanned using an Amersham™ 600 imager (GE Healthcare). Once imaged, blots could be stripped and reprobed for additional markers using other primary antibodies. For this method, membranes were stripped for 30 minutes at RT using Restore™ PLUS Western Blot Stripping Buffer from Thermo Scientific after first washing in PBS. Following this, membranes were washed in TBST and blocking with 5% milk in TBST for 1 hr at RT. The above blotting protocol was then followed for staining of total ZAP70. FIG. 4 demonstrates the pZAP70 protein level normalized to the ZAP70 intensity compared to control T-cells with 2-deoxy-2-fluoro-L-fucose-treated T-cells.

### Example 5

### Activated T-Cell Expression of Galectin-3

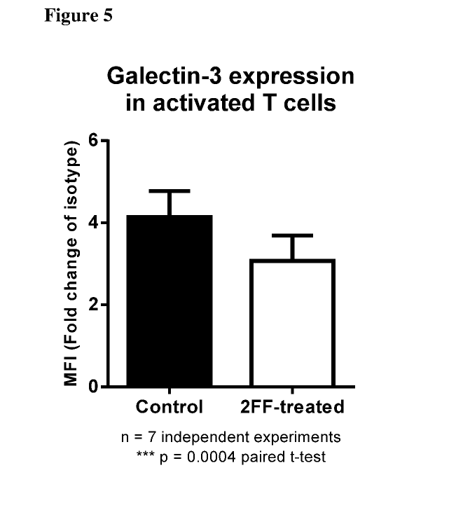

Purified T-cells were plated in 12-well plates (10⁶ cells/well, 1-2 mL T-cell medium) and were activated with CD3/CD28 beads (4 μL beads/2 mL media, Miltenyi Biotec). Cells were incubated at 37° C. overnight and were then analyzed for Galectin-3 expression after fixation and permeabilization followed by FACS analysis. In brief, cells were pelleted (2×10⁵ and washed twice with BD stain buffer (FBS) followed by resuspension in cold BD Cytofix™ fixation buffer (30 min, RT). Cells were pelleted, washed twice in BD stain buffer (FBS), and resuspended in BD Perm/Wash™ buffer (30 min, RT). Cells were washed into fresh BD Perm/Wash™ buffer and stained with anti-Galectin-3 antibody (Life Technologies, or with the appropriate isotype control) at RT (40 min, dark). Cells were washed twice in BD Perm/Wash™ at RT and resuspended in BD stain buffer (FBS) for analysis with an LSRII flow cytometer. FIG. 5 demonstrates the fold-change in the Galectin-3 MFI in the 2-deoxy-2-fluoro-L-fucose-treated T-cells compared to control T-cell cells.

The T-cell receptor (TCR) directly interacts with peptide-MHC on antigen presenting cells and is suggested to be a fucosylated molecule (Garcia et al. 1996, Science, 274). We observed an increase in EBV-specific tetramer binding by flow cytometry in 2-deoxy-2-fluoro-L-fucose-treated T-cells compared to control cells (slight increases were also observed with other antigens; flu, CMV). This is indicative of an increase in the affinity of TCR for p-MHC. In addition, the removal of antigen (peptide antigen in the form of serum proteins) from the co-culture of T-cells with DCs resulted in little to no increased activation markers on the DCs. These results taken together suggest that the TCR/pMHC interaction is being modulated by 2-deoxy-2-fluoro-L-fucose treatment. In order for the TCR to become engaged and T-cell signaling to occur, the TCR/peptide-MHC interaction must be strong enough to overcome galectin-glycoprotein lattice interactions which regulate basal signaling and activation of the TCR. We have observed that with 2-deoxy-2-fluoro-L-fucose treatment there is a reduction in galectin-3 levels in T-cells which would reduce the galectin-3-glycoprotein interactions and allow for easier TCR/pMHC-mediated signaling. When we examined TCR signaling we observed that Zap70, a protein known to be in the center of the TCR signaling cascade, is significantly more phosphorylated and remains phosphorylated for a longer period of time in 2-deoxy-2-fluoro-L-fucose-treated T-cells compared to naive cells (similar results were observed with other signaling proteins).

### Example 6

### TGFβ Signaling and Regulatory T-Cell Analysis

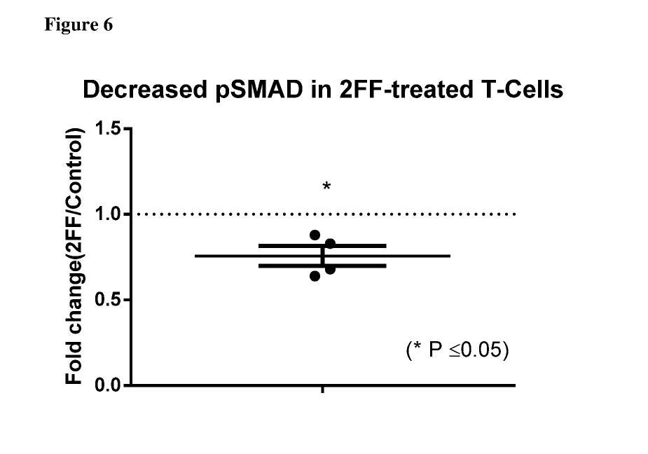

Cells were plated in 6-well dishes (2.5×10⁶ cells/well) with serum-free RPMI medium (1% PS, 2.5 mL) and after 6 h of serum starvation, cells were stimulated with TGFβ (5 ng/mL) for 10 min. The cells were harvested, washed twice in PBS, and snap frozen on dry ice followed by storage at −80 ° C. Cells were lysed with RIPA buffer (containing DNAse and protease/phosphatase inhibitors). As described previously, cell lysates were run on SDS-PAGE and examined by western blot on nitrocellulose membranes with anti-pSMAD2 and anti-actin antibodies. FIG. 6 demonstrates the ratio of pSMAD2 to actin intensity for control T-cells compared to the same ratio for 2-deoxy-2-fluoro-L-fucose-treated T-cells.

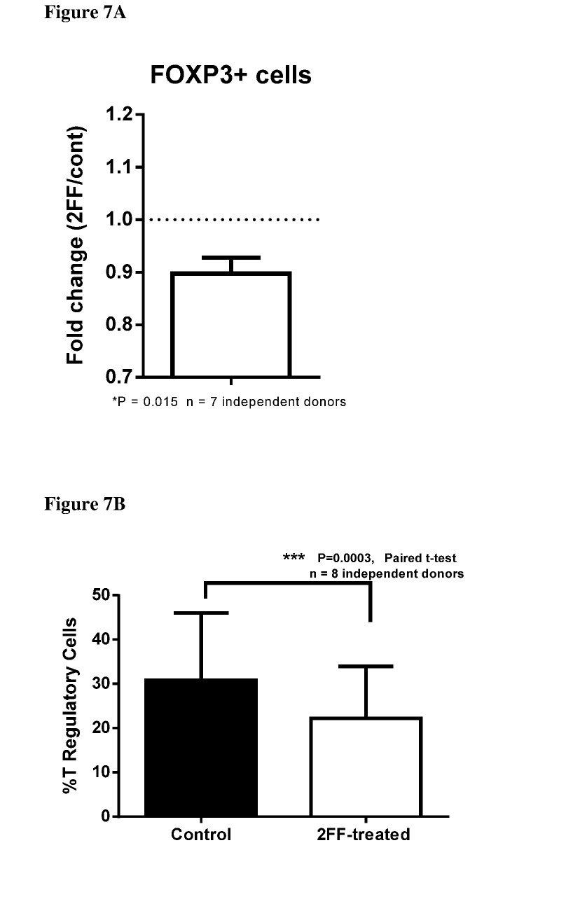

Regulatory T-cells in cultures were identified using the Human Regulatory T-cell Cocktail (BD Pharmingen™) and analyzed using FACS. Briefly, cells were washed into BD stain buffer (BSA) with human Fc block (EMD Millipore) and incubated on ice for 30 min. Cells were then stained with fluorescently labeled Human Regulatory T-cell Cocktail (BD Pharmingen™, 1:100 in BD stain buffer (BSA)) or with appropriate isotype controls (ice, dark, 40 min). Cells were then washed twice in BD stain buffer (BSA) and analyzed on an LSRII flow cytometer. Regulatory T-cells were identified as CD4+, CD25+ and CD127 LOW. FIGS. 7A and 7B demonstrate the percent of cells in this triple gated population.

T-cells were prepared in the same manner as for intracellular staining as above, where they were fixed and permeabilized and stained with an anti-FOXp3 antibody (BD Biosciences) and analyzed by FACS. In brief, after incubation in BD stain buffer (FBS) with Human Fc block (Miltenyi Biotec) on ice for 30 min as described previously, cells were stained with anti-CD4 antibody for 20 min in the dark (RT). An equal volume of BD stain buffer (FBS) was added and samples were spun and washed once in BD stain buffer (FBS) followed by resuspension in cold BD Cytofix™ fixation buffer for 30 min at RT. Cells were pelleted washed twice in BD stain buffer (FBS), resuspended, and incubated in BD Perm/Wash™ buffer at RT for 30 min. Cells were washed into fresh BD Perm/Wash™ buffer and stained with anti-FOXp3 antibody (or with the appropriate isotype control) at RT (45 min, dark). Cells were washed twice in BD Perm/Wash™ at RT and resuspended in BD stain buffer (FBS) for analysis with an LSRII flow cytometer. Cells were gated on CD4+ and from this gate FOXp3+ cells were identified. FIGS. 7A and 7B demonstrate the fold-change in the total number of FOXp3+ T-cells in the 2-deoxy-2-fluoro-L-fucose-treated culture compared to control T-cell culture.

Sprague-Dawley rats (CD®[Crl:CD®(SD)] were administered 2-deoxy-2-fluoro-L-fucose by oral gavage daily for 42 consecutive days at doses of 0, 125, 500 or 2000 mg/kg/day. Blood samples (0.4 mL/timepoint) were collected in sodium heparin at pretest and days 2, 7, 22, 43, 49, 56, and 71 of the study. Samples were processed within 24 hours of collection. Aliquots of the specimens were stained with predetermined volumes of previously tested and titered monoclonal antibodies specific for each marker. The red cells in each tube were lysed and after staining the cells were fixed with Versalyse Lysis Buffer solution (Beckman Coulter). The prepared specimens were analyzed on a Beckman-Coulter FC 500 MPL flow cytometer.

TGFβR is fucosylated and literature suggests that this fucosylation can affect TGFβ binding in tumor cells (British Journal of Cancer (2014) 110, 156-163). SMAD-mediated transcription requires phosphorylation following TGFβ binding to the TGFβI receptor. FIG. 6 demonstrates that 2-deoxy-2-fluoro-L-fucose treatment of T-cells results in reduced SMAD2 phosphorylation compared to control T-cells following TGFβ stimulation, supporting alterations in TGFβ binding and signaling on afucosylated T-cells. FOXp3 expression is driven by the SMAD transcription factors that are downstream from TGFβ signaling. 2-deoxy-2-fluoro-L-fucose-treated T-cells show decreased expression of FOXp3+ (FIG. 7A) which was associated with a decrease in regulatory T-cell populations (percent of total cells, FIG. 7B). Table 1 shows that this result was translated in vivo when rats were treated with 2-deoxy-2-fluoro-L-fucose. Beginning at the Day 2 collection, the relative proportion of regulatory T-cells (CD3+, CD4+, CD25+, FOXp3+) tended to decrease in all groups administered 2-deoxy-2-fluoro-L-fucose, relative to pretest values (Table 1). TGFβ signaling through phosphorylation of the SMAD2 transcription factor is diminished by 2-deoxy-2-fluoro-L-fucose treatment which in turn results in decreased FOXp3 expression in the T-cell culture and reduced numbers of T-regulatory cells. Reduction in regulatory T-cells is known to lead to a less immunosuppressive tumor microenvironment. Part of the activity conferred by anti-CTLA4 antibodies is directed depletion of regulatory T-cells (Simpson et al. (2013) JEM, 210). Anti-CTLA4 antibodies preclincially, and recently in the clinic, have been successfully combined with anti-PD1 or anti-PDL1 checkpoint antibodies (Larkin et al. (2015) NEJM 373). The effect of 2-deoxy-2-fluoro-L-fucose on reducing regulatory T-cells would suggest that combining 2-deoxy-2-fluoro-L-fucose affected T-cells with PD1 or PDL1 targeting agents would confer further enhenced anti-tumor immunity.

TABLE 1
Summary of Effects on Regulatory T-Cells^{a}

| | | 125 mg/ | | 500 mg/ | | 2000 mg/ | |
|---|---|---|---|---|---|---|---|
| | | kg/day | | kg/day | | kg/day | |
| Endpoint | Interval | M | F | M | F | M | F |
| Treg | Day 2 | −44% | −54% | −61% | −56% | −42% | −41% |
| % Gated | Day 7 | −64% | −67% | −63% | −60% | −43% | −39% |
| | Day 22 | −65% | −61% | −64% | −60% | −51% | −43% |
| | Day 43 | −59% | −64% | −58% | −50% | −29% | −28% |
| | Day 49^{b} | −46% | −40% | −48% | −43% | −6% | −19% |
| | Day 56^{b} | −47% | −51% | −47% | −38% | −12% | −16% |
| | Day 71^{b} | −42% | −45% | −48% | −40% | −13% | −26% |

M—Male;
F—Female
^{a}Percent change relative to pretest means
^{b}Recovery period

### Example 7

### Analysis of Secreted Cytokines in T-Cell/DC Co-Cultures and Observed In Vivo

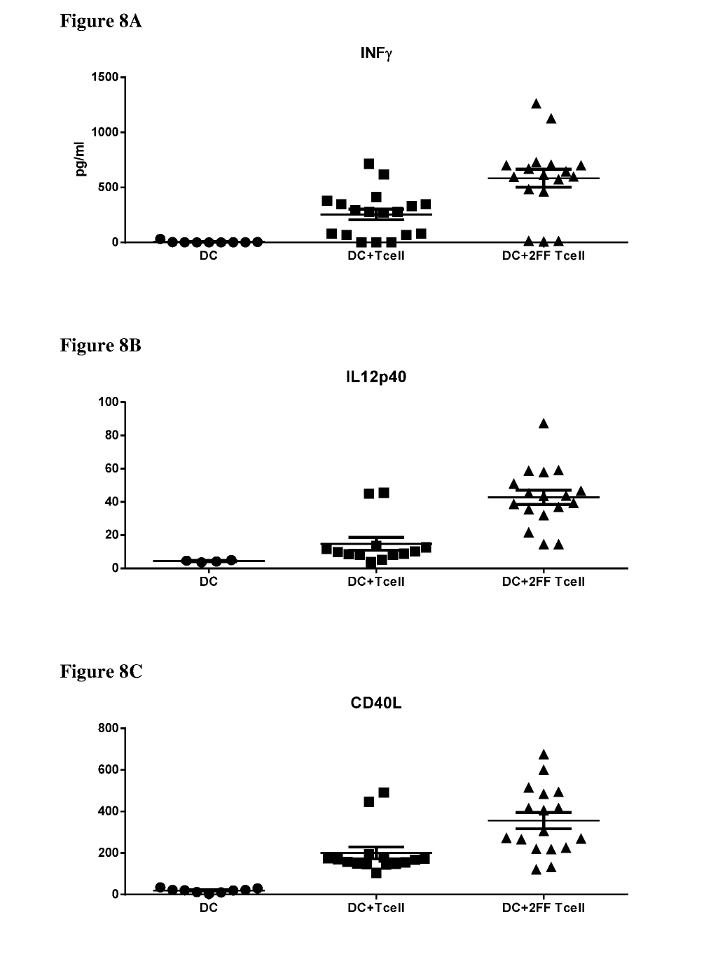

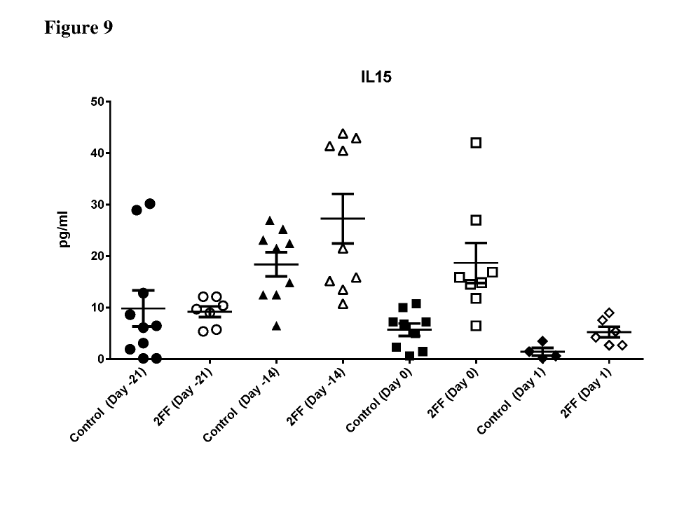

Tissue culture supernatants from T-cell/DC co-cultures described previously were collected and assessed for cytokine changes by Luminex assay. Similarly, blood samples from an A20 vaccine model study were allowed to clot and serum was collected for analysis of cytokines (MCYTOMAG01L1X-13 Mouse Cytokine Magnetic Bead Panel, Millipore). FIGS. 8A, 8B and 8C demonstrate that in the co-cultures of DCs and 2-deoxy-2-fluoro-L-fucose-treated T-cells, cytokines important for antigen specific T-cell activation such as INFy, IL12p40, and CD40L were increased in tissue culture supernatants when compared to cultures containing control T-cells. FIG. 9 demonstrates that in addition to cytokine changes observed in tissue culture co-cultures systems, specific increases in cytokines important for immune responses were observed in an A20 tumor vaccine model (described above, no depletion, samples taken at day-21, -14, 0, and 1; IL15). These data indicate that 2-deoxy-2-fluoro-L-fucose treatment can result in the upregulation of cytokines key for optimal immune responses in vitro and in vivo.

### Example 8

### In Vitro 2FF and Anti-PD1 Antibody Combination in Human PBMCs

CMV reactive human PMBCs from a CMV positive donor (Astarte™ Biologics) were thawed, plated into a 6-well plate and cultured overnight in DC media (30% DMEM 70% X-vivo+2 mM glutamax+10% ATB serum+1% PS). The following day the supernatant was collected to isolate T-cells and the adhering cells were washed in media and then replenished with 2 mL media supplemented with IL4 (100 ng/μL) and GMCSF (200 ng/μL) per well to differentiate and proliferate the adhering monocytes into DCs. The plate was incubated for 4-5 days and then cells were moved into a T25 flask. Each time the cells were passaged (every 2-3 days) new cytokines were given. The T-cells in the supernatant were isolated using StemSep human T-cell enrichment kit (Stemcell technologies). The T-cells were then resuspended in T-cell media (RPMI+10% Fetal calf serum, 1% PS) and spilt into two T25 flasks (with or without 100-200 μM 2FF). CD3CD28 antibody coated beads (Miltenyi Biotec) (20 μL/flask) were added to activate the T-cells. After 24 h IL2 was added (100 ng/μL). Each time the cells were passaged new IL2 and 2FF were added. The cells were cultured for 10-12 days and co-culture experiments were performed in DC media at a ratio of 10:1 (T-cells:DCs) in 48 well plates with or without 5 μg/mL CMV antigen stimulation (Astarte Biologics) and with or without 1 μg/mL anti-PD1 (Pembrolizumab, Keytruda). Co-cultures were incubated for 24 h after which supernatants were assayed for IFNγ and IL12p70 using the Human T-cell high sensitivity Lumenix assay (Millipore) following the manufacturer's instructions.

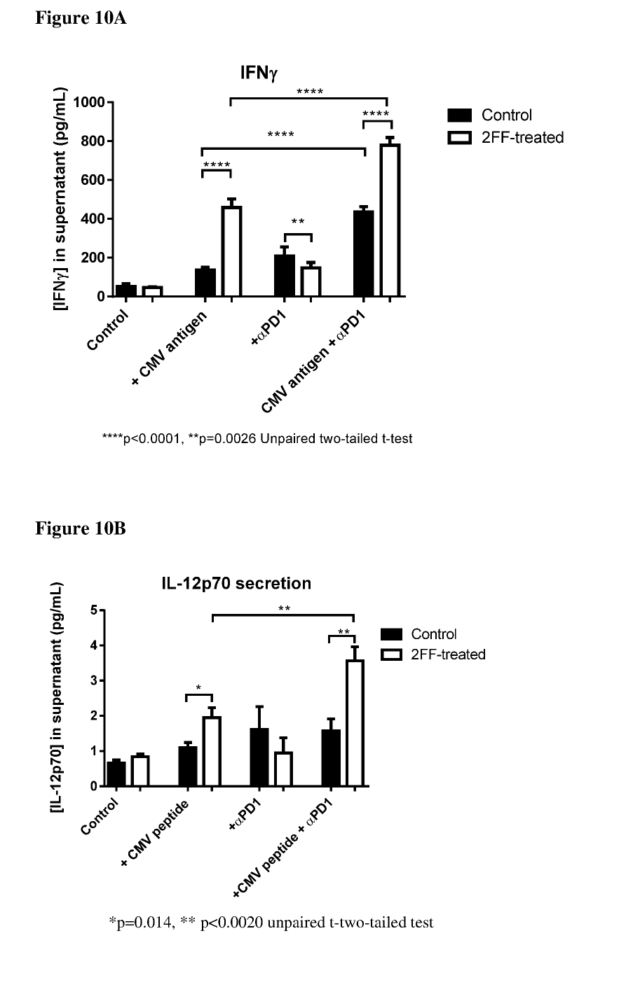

IFNγ and IL12p70 in the supernatants from 5 day co-cultures were measured using a Lumenix assay and the concentration of each cytokine was calculated using an internal standard curve. As shown in FIGS. 10A and 10B, 2FF matured T-cells exposed to CMV antigen have an elevated antigen specific T cell response compared to control cell and the addition of anti-PD1 to the combination further enhances the antigen specific response of 2FF matured T-cells.

### Example 9

### A20 Mouse Lymphoma Study

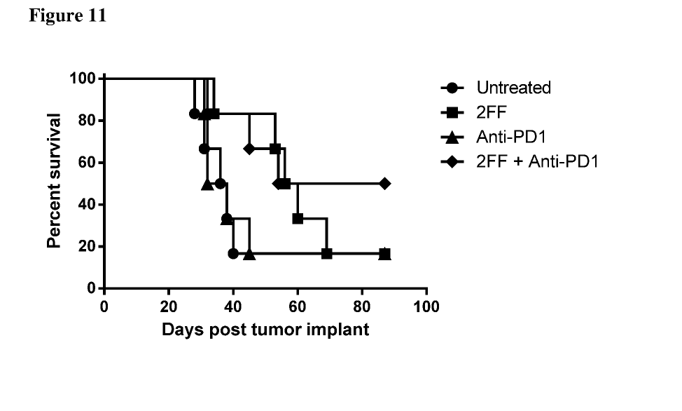

A20 cells (ATCC) were cultured in RPMI 1640 with 10% FBS, 10 mM HEPES, 1 mM sodium pyruvate, 50 μM 2-mercaptoethanol and penicillin (100 U/ml)/streptomycin (100 μg/ml) (PS). All mice (BALB/c, n=6/group) were implanted with 1×10⁵ A20 tumor cells (i.v.) and were divided into four groups: untreated; treated with 2FF only; treated with anti-PD1 antibody only; and treated with a combination of 2FF with anti-PD1 antibody. 2FF treatment groups received drinking water containing 20 mM 2FF beginning on the day of tumor implantand continued throughout the study. Mice not receiving 2FF were provided with normal drinking water. Anti-PD1 antibody (Ebiosciences, clone J43) treatment groups received three doses of 5 mg/kg every three days starting on day 5 post tumor implant. As shown in FIG. 11, the 2FF alone treated group had increased survival compared to the untreated group while the anti-PD 1antibody alone treated group did not improve survival compared to the untreated group. The group treated with the combination of 2FF with anti-PD1 antibody showed a durable survival of 50% of the mice by 54 days post tumor implant lasting through the end of the experiment at ˜90 days post tumor implant. This is compared to only 17% survival in the untreated and the anti-PD1 alone treated groups after about 40 days of treatment lasting to the end of the experiment, and also compared to only 17% survival in 2FF alone treated group after 70 days of treatment also lasting to the end of the experiment at ˜90 days. These data show that the combination of 2FF and anti-PD1 prolonged survival of tumor implanted mice compared to untreated and either 2FF or anti-PD1 single agent treatment.

### Discussion

As shown in the examples, 2FF has demonstrated the ability to reduce regulatory T-cell population, similar to what is observed with the anti-CTLA4 antibodies. The reduction in regulatory T-cell population is associated with attenuation of TGF-beta signaling and FOXp3 induction which is critical for regulatory T-cell development. Accordingly, combinatorial activity of 2FF in the presence of anti-PD1 blockade antibodies was evaluated both in vitro and in vivo. The examples have demonstrated that in vitro, 2FF cooperated with anti-PD1 antibody to increase antigen specific T-cell activation; and that in vivo, 2FF enhanced the anti-tumor response to anti-PD1 antibody and prolonged overall survival in a syngeneic mouse model of lymphoma compared to untreated or single agent treated groups.

### Example 10

### 4T1 Mouse Tumor Study

To evaluate the effect of 2FF treatment on tumor infiltrating immune cells balb/c mice were implanted with 4T1 cells (2×10⁴ injected sc.) and on the same day started on 20 mM 2FF drinking water. On day 19 (when tumors were approximately ˜250-300 mm³) or day 28 (when tumors were ˜800-1000 mm³) tumors were removed, finely minced with a razor blade and suspend in 5 mL cold disassociation buffer (DMEM (high glucose) media, 5% FBS, 1 M HEPES, 2 mg/mL collagenase D (Roche), 0.1 mg/mL DNAse I (50 mg/mL stock in 20 mM Tris-HCL (ph7.5), 1 mM MgCl₂, 50% glycerol) and transferred to a Miltenyi C-tube. GentleMACS was run on each tube and the tubes incubated at 37° C. for 40 minutes with continuous rotation. GentleMACS was then run twice and the tubes quickly spun down to collect the tumor in the bottom of the tube. The tube contents was then passed through a 70 μm mesh filter into a 50 mL falcon tube using the rubber stopper of a syringe to gently dissociate the tissue. The filter was washed with 5 mL of cold enzyme inhibitor buffer (DMEM (high glucose), 0.25 M EDTA) and the tube spun down, supernatant discarded and the samples re-suspend in PBS. Cells were then stained with eFluor®506 viability dye (eBioscience) following the manufactures instructions. Cells were washed twice in FACS staining buffer (0.5% FBS, 0.05% NaN3) and immune phenotyped with three different panels of antibodies, each containing an antibody against CD45 to identify immune cells. The cells phenotyped for dendritic cell antigens were stained with the desired antiboides for 30 minutes in the dark on ice, washed twice in staining buffer and resuspended in 1:4 PBS:fixation buffer (Biolgend) and stored at 4° C. overnight. The following day cells were washed in FACS stain buffer and analyzed on the LSRII flow cytometer. The cells phenotyped for T regulatory cells were first stained with the desired antibodies against cell surface antigens for 30 minutes in the dark on ice. The cells were then washed and fixed, permeabilized and stained with FOXp3 using the eBioscience staining set as per the manufactures instructions (catalog #77-5775).

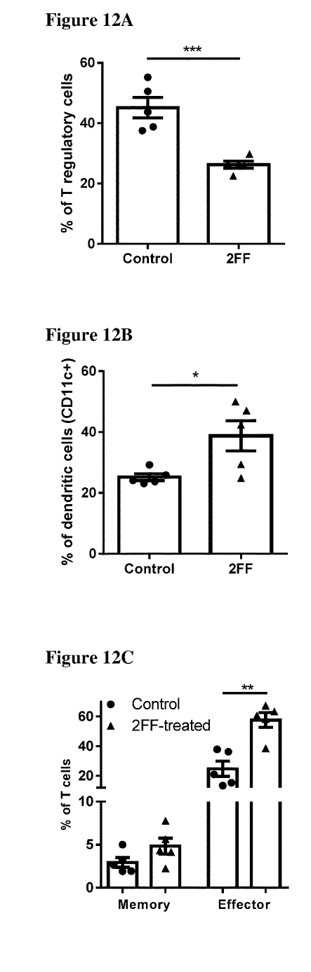

As shown in FIGS. 12A-C, 2FF-treated animals had significantly decreased T regulatory cells and increased dendritic cells which are more activated at day 19 after implantation. The results also show that at day 28 after implantation, tumors from 2FF-treated animals had increased memory and effector T cells.

1. A method for treating cancer or for inhibiting the proliferation of a tumor in a subject in need thereof comprising administering to the subject an effective amount of 2-deoxy-2-fluoro-L-fucose or a prodrug thereof, or a pharmaceutically acceptable salt thereof, in combination with a checkpoint inhibitor.

2. (canceled)

3. The method of claim 1 wherein the combination of 2-deoxy-2-fluoro-L-fucose or a prodrug thereof, or a pharmaceutically acceptable salt thereof, and the checkpoint inhibitor administered provides an additive or synergistic effect in the treatment of the cancer or in the inhibition of the proliferation of tumor cells.

4. The method of claim 3 wherein the combination of 2-deoxy-2-fluoro-L-fucose or a prodrug thereof, or a pharmaceutically acceptable salt thereof, and the checkpoint inhibitor administered provides a synergistic effect in the treatment of the cancer or in the inhibition of the proliferation of tumor cells.

5. The method of claim 1, wherein 2-deoxy-2-fluoro-L-fucose or a pharmaceutically acceptable salt thereof is administered in combination with a checkpoint inhibitor.

6. The method of claim 1, wherein the prodrug of 2-deoxy-2-fluoro-L-fucose or a pharmaceutically acceptable salt thereof is administered in combination with a checkpoint inhibitor.

7. The method of claim 6, wherein a carboxylic ester prodrug of 2-deoxy-2-fluoro-L-fucose or a pharmaceutically acceptable salt thereof is administered in combination with a checkpoint inhibitor.

8. The method of claim 7, wherein an acetate ester of 2-deoxy-2-fluoro-L-fucose or a pharmaceutically acceptable salt thereof is administered in combination with a checkpoint inhibitor.

9. (canceled)

10. The method of claim 1, wherein the checkpoint inhibitor is selected from the group consisting of a monoclonal antibody, a humanized antibody, a fully human antibody and a fusion protein or a combination thereof.

11. The method of claim 10, wherein the checkpoint inhibitor inhibits or interacts with a ligand of a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN -15049, CHK 1, CHK2, A2aR, and B-7 family ligands or a combination thereof.

12. The method of claim 11, wherein the checkpoint inhibitor is a PD-L1, PD-L2, or PD-1 inhibitor.

13. The method of claim 12, wherein the checkpoint inhibitor is a PD-1 inhibitor.

14. The method of claim 13, wherein the checkpoint inhibitor is a PD-1 inhibitor is nivolumab or pembrolizumab.

15. (canceled)

16. The method of claim 1, wherein the cancer is selected from the group consisting of urogenital, gynecological, lung, gastrointestinal, head and neck cancer, brain cancers including malignant gliomas and brain metastases, malignant mesothelioma, non-metastatic or metastatic breast cancer, malignant melanoma, Merkel Cell Carcinoma or bone and soft tissue sarcomas, haematologic neoplasias, multiple myeloma, lymphomas such as Hodgkin's disease, non-Hodgkin's lymphoma, acute myelogenous leukemia, chronic myelogenous leukemia, myelodysplastic syndrome and acute lymphoblastic leukemia, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

17. The method of claim 16, wherein the cancer is non-small cell lung cancer (NSCLC), breast cancer, or colorectal cancer.

18. The method of claim 17, wherein the cancer is non-small cell lung cancer (NSCLC).

19. The method of claim 1, wherein a composition comprising 2-deoxy-2-fluoro-L-fucose or a prodrug thereof, or a pharmaceutically acceptable salt thereof, is administered to the subject.

20. The method of claim 19, wherein the composition is a solid or a liquid formulation.

21. The method of claim 1, wherein the subject is a mammal.

22. The method of claim 21, wherein the mammal is a human.

23. A method for initiating, enhancing or prolonging the effects of a checkpoint inhibitor, or enabling a subject to respond to a checkpoint inhibitor in a subject in need thereof comprising administering to the subject an effective amount of 2-deoxy-2-fluoro-L-fucose or a prodrug thereof, or a pharmaceutically acceptable salt thereof, in combination with a checkpoint inhibitor.

24. The method of claim 23, wherein the combination of 2-deoxy-2-fluoro-L-fucose or a prodrug thereof, or a pharmaceutically acceptable salt thereof, and the checkpoint inhibitor administered provides an additive or synergistic effect in the treatment of the cancer or in the inhibition of the proliferation of tumor cells.

25. The method of claim 24, wherein the combination of 2-deoxy-2-fluoro-L-fucose or a prodrug thereof, or a pharmaceutically acceptable salt thereof, and the checkpoint inhibitor administered provides a synergistic effect in the treatment of the cancer or in the inhibition of the proliferation of tumor cells.

26. The method of claim 23, wherein the anti-tumor response is selected from inhibiting tumor growth, inducing tumor cell death, tumor regression, preventing or delaying tumor recurrence, tumor growth, tumor spread and tumor elimination.

27. The method of claim 23, wherein 2-deoxy-2-fluoro-L-fucose or a pharmaceutically acceptable salt thereof is administered in combination with a checkpoint inhibitor.

28. The method of claim 23, wherein the prodrug of 2-deoxy-2-fluoro-L-fucose or a pharmaceutically acceptable salt thereof is administered in combination with a checkpoint inhibitor.

29. The method of claim 28, wherein a carboxylic ester prodrug of 2-deoxy-2-fluoro-L-fucose or a pharmaceutically acceptable salt thereof is administered in combination with a checkpoint inhibitor.

30. The method of claim 29, wherein an acetate ester of 2-deoxy-2-fluoro-L-fucose or a pharmaceutically acceptable salt thereof is administered in combination with a checkpoint inhibitor.

31. (canceled)

32. The method of claim 23, wherein the checkpoint inhibitor is selected from the group consisting of a monoclonal antibody, a humanized antibody, a fully human antibody and a fusion protein or a combination thereof.

33. The method of claim 32, wherein the checkpoint inhibitor inhibits or interacts with a ligand of a checkpoint protein selected from the group consisting of CTLA-4, PD-1, PD-L1, PD-L2, B7-H3, B7-H4, BMA, HVEM, TIM3, GAL9, LAG3, VISTA, KIR, 2B4, CD160, CGEN -15049, CHK 1, CHK2, A2aR, and B-7 family ligands or a combination thereof.

34. The method of claim 33, wherein the checkpoint inhibitor is a PD-L1, PD-L2, or PD-1 inhibitor.

35. The method of claim 34, wherein the checkpoint inhibitor is a PD-1 inhibitor.

36. The method of claim 35, wherein the checkpoint inhibitor is a PD-1 inhibitor is nivolumab or pembrolizumab.

37. (canceled)

38. The method of claim 23, wherein the cancer is selected from the group consisting of urogenital, gynecological, lung, gastrointestinal, head and neck cancer, brain cancers including malignant gliomas and brain metastases, malignant mesothelioma, non-metastatic or metastatic breast cancer, malignant melanoma, Merkel Cell Carcinoma or bone and soft tissue sarcomas, haematologic neoplasias, multiple myeloma, lymphomas such as Hodgkin's disease, non-Hodgkin's lymphoma, acute myelogenous leukemia, chronic myelogenous leukemia, myelodysplastic syndrome and acute lymphoblastic leukemia, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

39. The method of claim 38, wherein the cancer is non-small cell lung cancer (NSCLC), breast cancer, or colorectal cancer.

40. The method of claim 39, wherein the cancer is non-small cell lung cancer (NSCLC).

41. The method of claim 23, wherein a composition comprising 2-deoxy-2-fluoro-L-fucose or a prodrug thereof, or a pharmaceutically acceptable salt thereof, is administered to the subject.

42. The method of claim 41, wherein the composition is a solid or a liquid formulation.

43. The method of claim 23, wherein the subject is a mammal.

44. The method of claim 43, wherein the mammal is a human.
